(19) United States
(12) Patent Application Publication
(10) Pub. No.: US 2011/0218554 A1
(43) Pub. Date: Sep. 8, 2011

(54) Automatic clip applier for clamping two ligating clips simultaneously

(75) Inventors: Ming-Huei Cheng, Taipei City (TW); Steve L. Henry, Taipei City (TW)

(73) Assignees: Ming-Huei CHENG, Taipei City (TW); Steve L. HENRY, Taipei City (TW); Holger ENGEL, Taipei City (TW)

(21) Appl. No.: 12/801,702

(22) Filed: Jun. 22, 2010

(30) Foreign Application Priority Data
Mar. 3, 2010 (TW) 099106088

(51) Int. Cl. A61B 17/10

(52) U.S. Cl. 606/143

(57) ABSTRACT

An automatic clip applier for clamping ligating clips is disclosed, which includes: a handling unit, and two clamping means laminating with each other and connecting to the handling unit. Each clamping means respectively contains: a jaw unit containing a clipping portion to clamp a ligating clip; a magazine unit for storing plural ligating clip; a feeding unit for translating the endmost ligating clip from the magazine unit to the clipping portion of the jaw unit; and a dispensing unit for serially translating the endmost ligating clip to the end of the feeding unit. Accordingly, the automatic clip applier provided by the present invention can be used for clamping two ends of a vessel simultaneously, and the step of taking ligating clips can also be omitted. Hence, the surgical procedure can be simplified and the surgical time can be shortened by use of the automatic clip applier of the present invention.

### BACKGROUND OF THE INVENTION

1. Field of the Invention

The present invention relates to an automatic clip applier for clamping two ligating clips simultaneously and, more particularly, to an automatic clip applier which can be used to clamp two ligating clips simultaneously and cut a blood vessel.

2. Description of Related Art

During a surgical operation, a ligating of the blood vessels is usually applied to prevent bleeding of blood vessels, and some tools for such operations have been developed to assist the ligation of the blood vessels. Commonly, for example, a clip applier is used to pick up a ligating clip, and then the clip applier is clamped to deform the ligating clips to close the blood vessels. In order to save time in picking up the ligating clip, automatic clip appliers, which can provide plural ligating clips serially, are commercially available.

An automatic clip applier is a clip applier, which has plural ligating clips stored therein and transmits the ligating clips sequentially to a clipping portion. Hence, a surgeon can use the automatic clip applier to close blood vessels, and does not have to pick the ligating clips up. More specifically, during the process of closing a blood vessel, the ligation clip positioned at the clipping portion is clamped to close one end of the blood vessel, then another ligating clip is transmitted automatically to the clipping portion for closing the other end of the blood vessel. Hence, the surgeon does not have to pick up another ligating clip, and can clamp the automatic clip applier directly to close the other end of the blood vessel.

Although the step of picking up ligating clip can be omitted through using an automatic clip applier, there are still many complex steps such as a tool exchanging step can be simplified during the operation. For example, the automatic clip applier has to be operated two times for clamping the ligating clips to close two ends of the blood vessel, which is going to be cut. Then, a cutting tool such as a surgical blade is picked up to cut the blood vessel at an appropriate position. Hence, even though the automatic clip applier is used during the surgical performance, the step of clamping the ligating clips still has to be performed two times and the cutting tool has to be exchanged.

Therefore, it is desirable to provide an automatic clip applier, which can close two ends of a blood vessel at a time. In addition, it is also desirable to provide an automatic clip applier with a function of cutting the blood vessel, in order to simplify the surgical process and save time.

### SUMMARY OF THE INVENTION

The object of the present invention is to provide an automatic clip applier for clamping two ligating clips simultaneously, which can close two ends of the blood vessel simultaneously in a single clamping step. Therefore, the operation process can be simplified and the surgical time can be reduced.

To achieve the object, the automatic clip applier for clamping ligating clips comprises: a handling unit; and two clamping means laminating with each other and connecting to the handling unit. Each clamping means respectively comprises: a jaw unit containing a clipping portion to clamp a ligating clip; a jaw housing containing a cavity, wherein the jaw unit is disposed in the cavity of the jaw housing; a magazine unit for storing plural ligating clips; a feeding unit for moving the endmost ligating clip from the magazine unit to the clipping portion of the jaw unit; a dispensing unit for serially translating the endmost ligating clip to a front end of the feeding unit; and an operation unit connecting to the handling unit, the feeding unit, and the jaw housing, wherein the jaw housing is pushed and the feeding unit is drawn by operating the handling unit to move the jaw housing toward the clipping portion of the jaw unit to clamp the ligating clip positioning at the clipping portion, and to move the feeding unit in an opposite direction of the movement of the jaw housing, and the feeding unit moves back by restoring the handling unit to deliver the endmost ligating clip to the clipping portion of the jaw unit.

According to the automatic clip applier for clamping ligating clips of the present invention, the handling unit connects to two clamping means, and each jaw units of the two clamping means respectively clamps a ligating clip. Hence, when a surgeon operates the handling unit, one or more ligating clips can be clamped simultaneously, and it is possible to close two ends of the blood vessel in a single clamping step. In addition, the automatic clip applier for clamping ligating clips of the present invention comprises a magazine unit for storing plural ligating clips, and the ligating clips can be moved to the jaw unit through the feeding unit. Hence, the feeding unit can move the ligating clips to the clipping portion, so the step of picking up the ligating clips can be omitted during the operation process.

The automatic clip applier for clamping ligating clips of the present invention may further comprise: a cutting unit and an advancing unit, wherein, the cutting unit connects to the advancing unit, and the cutting unit is moved toward the clipping portions of the jaw units by operating the advancing unit. Preferably, one end of the cutting unit corresponds to an interval between the clipping portions of the two clamping means. After the cutting unit is moved, the cutting unit can cut a blood vessel ligated with the ligating clips. Herein, the structure of the advancing unit is not particularly limited, as long as the advancing unit can move the cutting unit toward the clipping portion of the jaw unit.

Hence, after the two ends of the blood vessel are ligated by use of the automatic clip applier of the present invention, the advancing unit of the automatic clip applier of the present invention can be used to move the cutting unit toward a predetermined cutting position of the blood vessel to cut the blood vessel. Therefore, the step of picking up the cutting unit can be omitted, so the operation process can be simplified and the surgical time can be reduced.

According to one aspect of the present invention, the cutting unit and the advancing unit may be disposed between the two clamping means. In addition, the automatic clip applier for clamping ligating clips may further comprise a channel cavity disposed between the two clamping means, and the advancing unit is placed inside the channel cavity.

According to another aspect of the present invention, the cutting unit and the advancing unit may be disposed on a side of one of the clamping means. In addition, the automatic clip applier for clamping ligating clips may further comprise a fixing unit disposed on the side of one of the clamping means, wherein the fixing unit has a channel cavity, and the advancing unit is placed inside the channel cavity.

Furthermore, according to the automatic clip applier for clamping ligating clips of the present invention, the cutting unit may comprise a cutting component and a connecting component, wherein the cutting component connects with the connecting component, and the connecting component connects with the advancing unit. In addition, the cutting component may be a blade or a hook.

The automatic clip applier for clamping ligating clips of the present invention may further comprise an elastic element, wherein the connecting component is sheathed into the elastic element, and one end of the elastic element connects to the advancing unit. Hence, after the applied force on the advancing unit is removed, the elastic element can provide a restoring force to return the cutting unit and the advancing unit to its original state without a force applied thereon. Herein, the shape of the elastic element is not particularly limited, as long as it can provide a restoring force. For example, the elastic element can be a spring.

In addition, according to the automatic clip applier for clamping ligating clips of the present invention, the operation unit may further comprise: a pull component and a push component. The pull component connects with the feeding unit and the handling unit and the push component connects with the jaw housing and the handling unit. When the push component is pushed and the pull component is drawn by operating the handling unit, the pushing force applied on the push component can move the jaw housing toward the clipping portion of the jaw unit to clamp the ligating clip positioned at the clipping portion, and the drawing force applied on the pull component can move the feeding unit in an opposite direction of the movement of the jaw housing. When the feeding unit is moved back by restoring the handling unit, the endmost ligating clips in the magazine unit can be delivered to the clipping portion of the jaw unit.

Furthermore, according to the automatic clip applier for clamping ligating clips of the present invention, each jaw unit may further comprise an elongation part connecting with the clipping portion. The elongation part connects with the clipping portion with an angle, and the degree of the angle is not particularly limited. Preferably, the degree of the angle is 90° to 180°. More preferably, the degree of the angle is 120° to 150°. In addition, in two jaw units of the clamping means, the angles between the elongation parts and the clipping portions may be the same or different. Preferably, the angles between the elongation parts and the clipping portions of the two clamping means are the same. Hence, the jaw units can be in a straight state or a curved state, according to the requirement for the operation. For example, when a blood vessel locates in a deeper position, the automatic clip applier with a jaw unit in a curved state may be selectively used for clamping the blood vessel. Therefore, during the clamping process, the blood vessel can be easily clamped and cut with the end extended from the jaw unit in a curved state.

When the automatic clip applier for clamping ligating clips of the present invention is used during the operation, two ligating clips can be clamped simultaneously to ligate two ends of a blood vessel in a single clamping step. However, when a conventional automatic clip applier is used, the clamping step has to be performed two times to ligate two ends of a blood vessel. Hence, the operation process can be simplified and the surgical time can be shortened when the automatic clip applier of the present invention is used. In addition, the automatic clip applier of the present invention further comprises a cutting unit, which can be moved toward the cutting region of a blood vessel by applying a force on an advancing unit, to cut the blood vessel. Compared to the operation process using the conventional automatic clip applier without a cutting unit, the step of picking up the cutting unit can be omitted when the automatic clip applier of the present invention is used. Therefore, the operation process can further be simplified and the surgical time can be shortened when the automatic clip applier with the cutting unit of the present invention is used. Furthermore, the automatic clip applier of the present invention further comprises a magazine unit, a feeding unit and a dispensing unit, so ligating clips stored in the magazine unit can be moved to the clipping portions of the jaw units through the feeding unit and the dispensing unit. Hence, the process of getting the ligating clips can be omitted, so the surgical time can be shortened.

### BRIEF DESCRIPTION OF THE DRAWINGS

FIG. 1 is a perspective view of the automatic clip applier for clamping two ligating clips of Embodiment 1 of the present invention;

FIG. 2 is an exploded view of a clamping housing of the automatic clip applier for clamping two ligating clips of Embodiment 1 of the present invention;

FIG. 3 is an exploded view of an operating housing of the automatic clip applier for clamping two ligating clips of Embodiment 1 of the present invention;

FIG. 4 is an exploded view of a clamping mean of the automatic clip applier for clamping two ligating clips of Embodiment 1 of the present invention;

FIG. 5 is an operational view of the automatic clip applier, partly removed for clarity, for clamping two ligating clips of Embodiment 1 of the present invention;

FIG. 6 is a cross-sectional view of the cutting mechanism of the automatic clip applier for clamping two ligating clips of Embodiment 1 of the present invention;

FIGS. 7A to 7C are cross-sectional views of the actuating mechanism of the feeding unit of the automatic clip applier for clamping two ligating clips of Embodiment 1 of the present invention;

FIG. 8 is a partial perspective view of the automatic clip applier for clamping two ligating clips of Embodiment 2 of the present invention;

FIG. 9 is a perspective view of the arrangement of the fixing unit, the advancing unit and the elastic element of the automatic clip applier for clamping two ligating clips of Embodiment 2 of the present invention;

FIG. 10 is a cross-sectional view of the jaw unit of the automatic clip applier for clamping two ligating clips of Embodiment 1 of the present invention;

FIG. 11 is a cross-sectional view of the jaw unit of the automatic clip applier for clamping two ligating clips of Embodiment 3 of the present invention; and

FIG. 12 is a cross-sectional view of the jaw unit of the automatic clip applier for clamping two ligating clips of Embodiment 4 of the present invention.

### DETAILED DESCRIPTION OF THE PREFERRED EMBODIMENT

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Embodiment 1

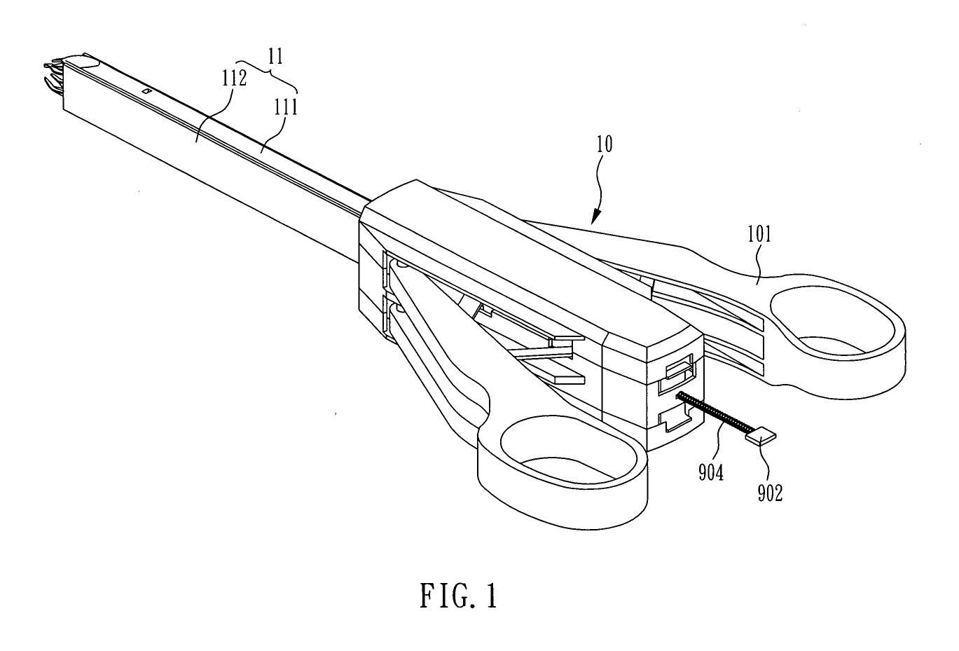

As shown in FIG. 1, the automatic clip applier for clamping two ligating clips of the present embodiment comprises: an operating housing 10, a pair of handling units 101, and a clamping housing 11. The clamping housing 11 longitudinally extends from the operating housing 10, and the handling units 101 also extend from the operating housing 10. The automatic clip applier of the present embodiment comprises two sets of clamping means, which are the same and correspond to each other. In order to clearly describe the automatic clip applier of the present embodiment, only one set of the clamping means is illustrated herein.

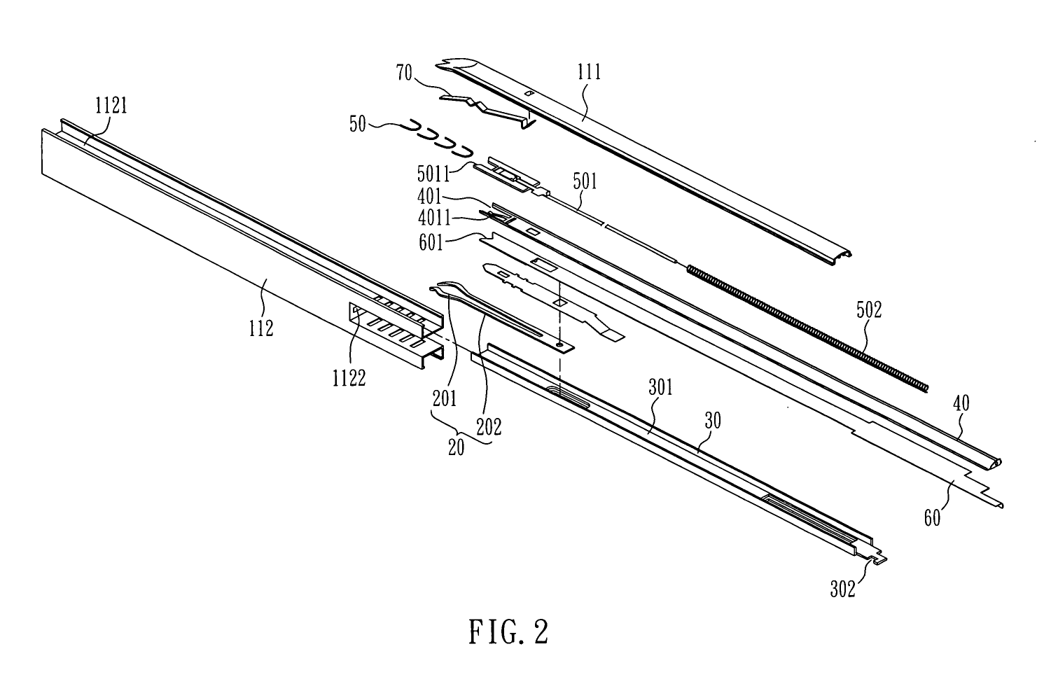

As shown in FIG. 1 and FIG. 2, the clamping housing 11 of the automatic clip applier of the present embodiment comprises: two upper caps 111 and a fixing frame 112. Each side of the fixing frame 112 respectively has a recess 1121, and the upper caps 111 are mounted on two sides of the fixing frame 122 to cover the recesses 1121. Herein, the jaw unit 20, the jaw housing 30, the magazine unit 40, the feeding unit 60, the dispensing unit 70, and the ligating clips 50 are disposed in the recesses 1121.

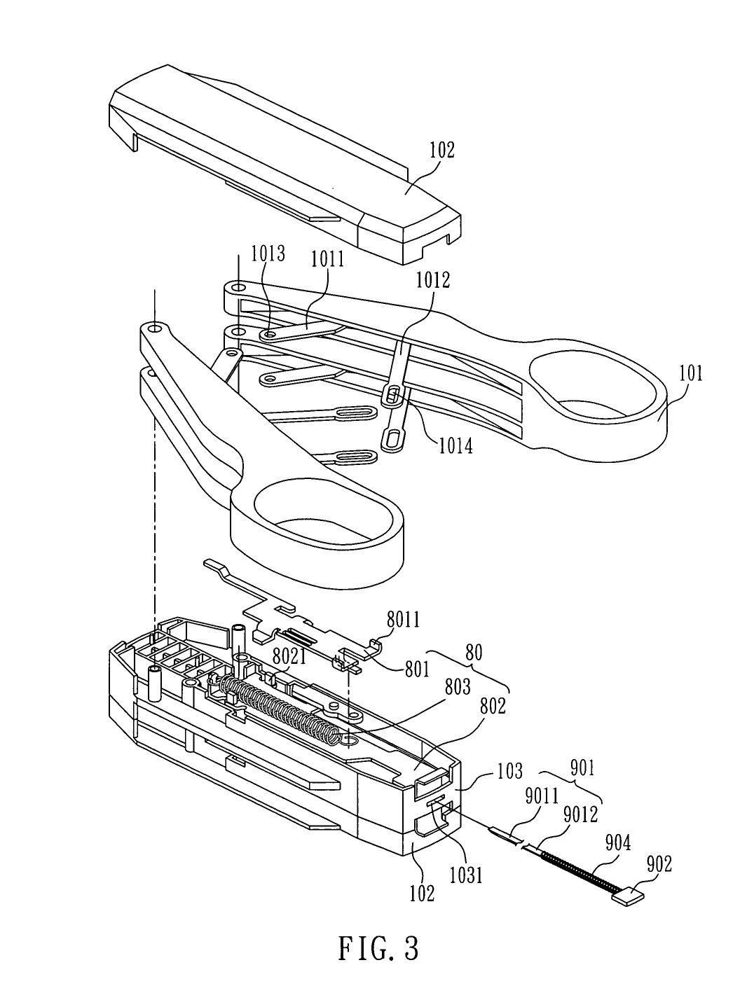

As shown in FIG. 1 and FIG. 3, the operating housing 10 of the automatic clip applier of the present embodiment comprises: two coverings 102 and a fixing frame 103. The fixing frame 103 is disposed between two coverings 102, and the operation unit 80 is disposed on the fixing frame 103 and placed in the space between the coverings 102 and the fixing frame 103. The handling unit 101 pivots on the operating unit 80 through a first lever 1011 and a second lever 1012. More specifically, the operation unit 80 further comprises: a pull component 801 and a push component 802. The pull component 801 comprises a pivot knob 8011 received in the hole 1014 of the second lever 1012, and the push component 802 also comprises a pivot knob 8021 received in the hole 1013 of the first lever 1011. Hence, when the handling unit 101 is operated, the push component 802 can be pushed and the pull component 801 can be drawn.

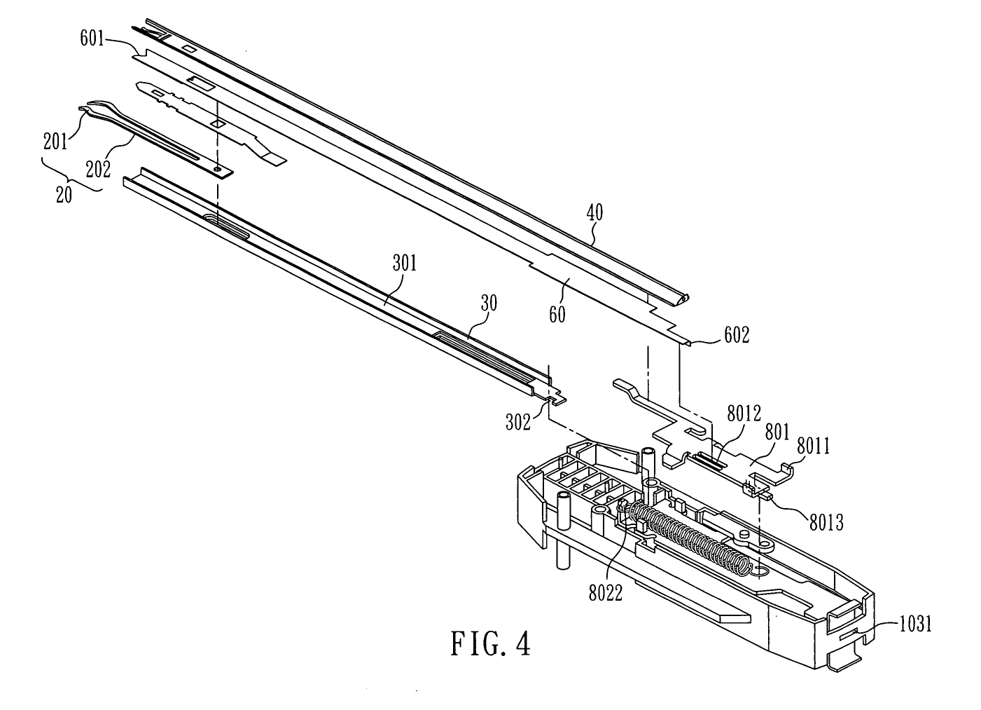

As shown in FIG. 2, FIG. 3, and FIG. 4, the automatic clip applier of the present embodiment comprises: a handling unit 101, and two clamping means laminating with each other and connecting to the handling unit 101. Each clamping means respectively comprises: a jaw unit 20 containing a clipping portion 201 to clamp a ligating clip 50; a jaw housing 30 containing a cavity 301, wherein the jaw unit 20 is disposed in the cavity 301 of the jaw housing 30; a magazine unit 40 for storing plural ligating clips 50; a feeding unit 60 for moving the endmost ligating clip 50 from the magazine unit 40 to the clipping portion 201 of the jaw unit 20; a dispensing unit 70 for serially translating the endmost ligating clip 50 to the end 601 of the feeding unit 60; and an operation unit 80 connecting to the handling unit 101, the feeding unit 60, and the jaw housing 30. When a force is applied on the handling unit 101, the jaw housing 30 is pushed and moved toward the clipping portion 201 of the jaw unit 20 to clamp the ligating clip 50 positioning at the clipping portion 201, and the feeding unit 60 is drawn and moved in an opposite direction of the movement of the jaw housing 30. When the force applied on the handling unit 101 is removed, the feeding unit 60 is moved back to deliver the endmost ligating clip 50 to the clipping portion 201 of the jaw unit 20.

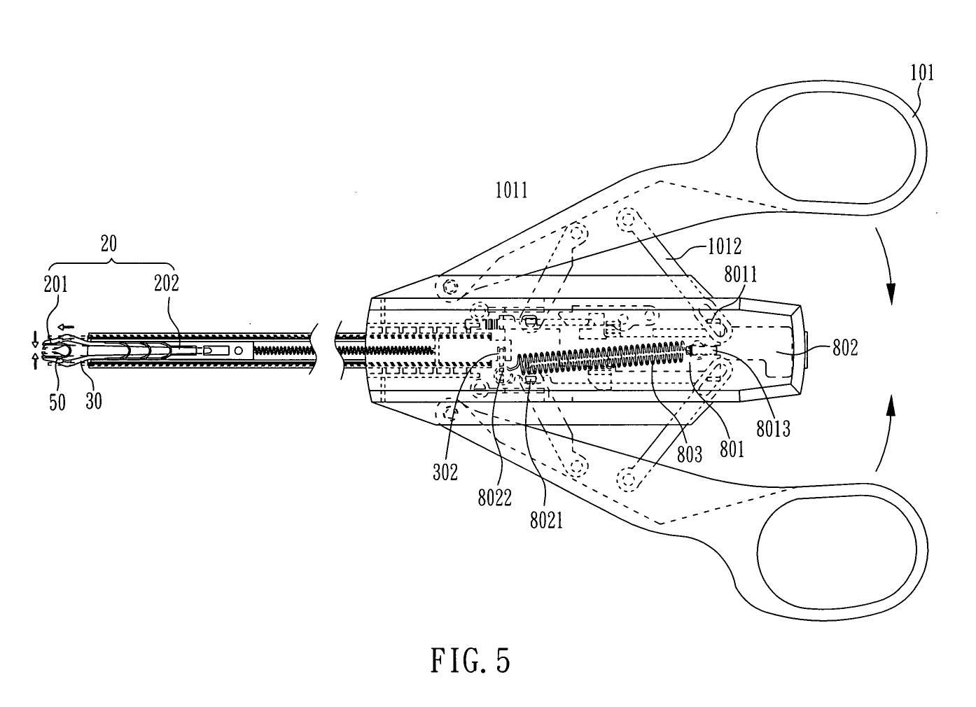

Hereafter, the actuating mechanism of the automatic clip applier of the present embodiment is detailed described. As shown in FIG. 3, FIG. 4 and FIG. 5, the jaw housing 30 of the automatic clip applier of the present embodiment further comprises a notch 302, which is adapted to a fixing knob 8022 of the push component 802 to connect the push component 802 with the jaw housing 30. In addition, the feeding unit 60 further comprises a fixing part 602, which is adapted into an opening 8012 of the pull component 801 to connect the pull component 801 with the feeding unit 60.

When the handling unit 101 is operated, the push component 802 is pushed through the first lever 1011, and the pull component 801 is drawn through the second lever 1012. The notch 302 of the jaw housing 30 is adapted to the fixing knob 8022 of the push component 802, so the jaw housing 30 is moved toward the clipping portion 201 of the jaw unit 20 to clamp the ligating clip (not shown in the figure) positioned at the clipping portion 20 when the handling unit 101 pushes the push component 802. In addition, the fixing part 602 is adapted to the opening 8012 of the pull component 801, so the feeding unit 60 can be moved in an opposite direction of the movement of the jaw housing 30 when the handling unit 101 draws the pull component 801.

As shown in FIG. 4 and FIG. 5, the operation unit 80 of the automatic clip applier of the present embodiment further comprises an elastic element 803, wherein one end of the elastic element 803 is mounted on the fixing knob 8022 of the push component 802, and the other end is mounted on the fixing part 8013 of the pull component 801. Therefore, the elastic element 803 can provide a restoring force to return the pull component 801 and the push component 802 back to their original state that force is not applied thereon.

As shown in FIG. 2 and FIG. 3, the automatic clip applier of the present embodiment further comprises: a cutting unit 901 and an advancing unit 902, wherein the cutting unit 901 connects with the advancing unit 902, and the cutting unit 901 and the advancing unit 902 are arranged between the clamping means. More specifically, the fixing frames 112, 103 respectively comprise channel cavities 1122, 1031, wherein the channel cavities 1122, 1031 are arranged between two clamping means, and the advancing unit 902 is installed in the channel cavities 1122, 1031. When force is applied on the advancing unit 902, the cutting unit 901 is moved toward the clipping portion 201 of the jaw unit 20. Also, the cutting unit 901 and the advancing unit 902 are installed in the channel cavities 1122, 1031 of the fixing frames 112, 103, so the shift of the cutting unit 901 and the advancing unit 902 are limited in the space of the channel cavities 1122, 1031. Therefore, the cutting unit 901 and the advancing unit 902 can be shifted in a straight route.

As shown in FIG. 2 and FIG. 3, the cutting unit 901 further comprises: a cutting component 9011 and a connecting component 9012, wherein the cutting component 9011 connects with the connecting component 9012, and the connecting component 9012 connects with the advancing unit 902. In addition, the automatic clip applier of the present embodiment further comprises an elastic element 904, wherein the connecting component 9012 is sheathed in the elastic element 904, and one end of the elastic element 904 connects with the advancing unit 902. In the present embodiment, the cutting component 9011 is a blade.

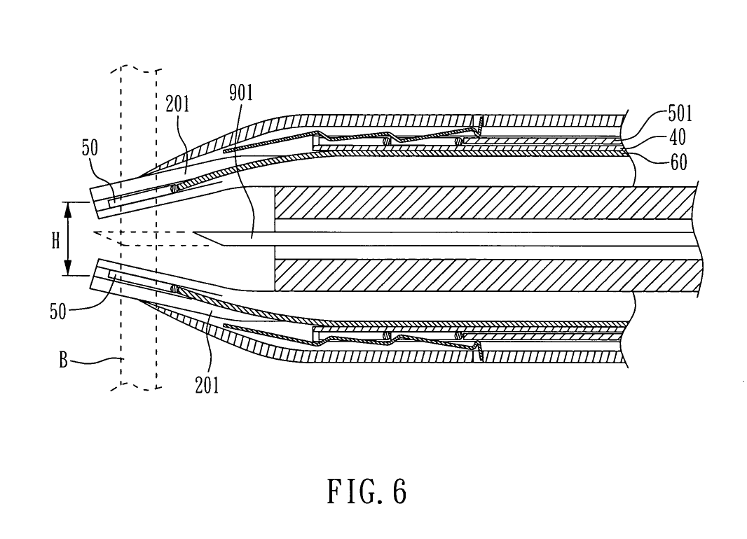

As shown in FIG. 1, FIG. 3 and FIG. 6, when the automatic clip applier of the present embodiment is applied for cutting the blood vessel B at the cutting position B3, the handling unit 10 is operated for clamping the ligating clips on the clipping portions 201 to ligate two ends of the blood vessel B. Then, the advancing unit 902 is pressed to compress the elastic element 904, so the advancing unit 902 can move the cutting unit 901 toward the cutting position B3. The cutting component 9011 of the cutting unit 901 corresponds to the interval H between two clip portions 201, so the cutting component 9011 can cut the blood vessel B ligated with the ligating clips 50 after the cutting unit 901 is pushed by the advancing unit 902.

As shown in FIG. 2, the automatic clip applier of the present embodiment further comprises: a tab 501 and an elastic element 502, wherein the tab 501 and the elastic element 502 are arranged in the magazine unit 40, plural ligating clips 50 are arranged in the magazine unit 40 and connect with a front end 5011 of the tab 501, and the tab 501 is sheathed in the elastic element 502. The position of the tab 501 in the magazine unit 40 can be adjusted through the elastic element 502. Hence, the storage space of the magazine unit 40 for storing the ligating clips 50 can be adjusted according to the amount of the ligating clips 50.

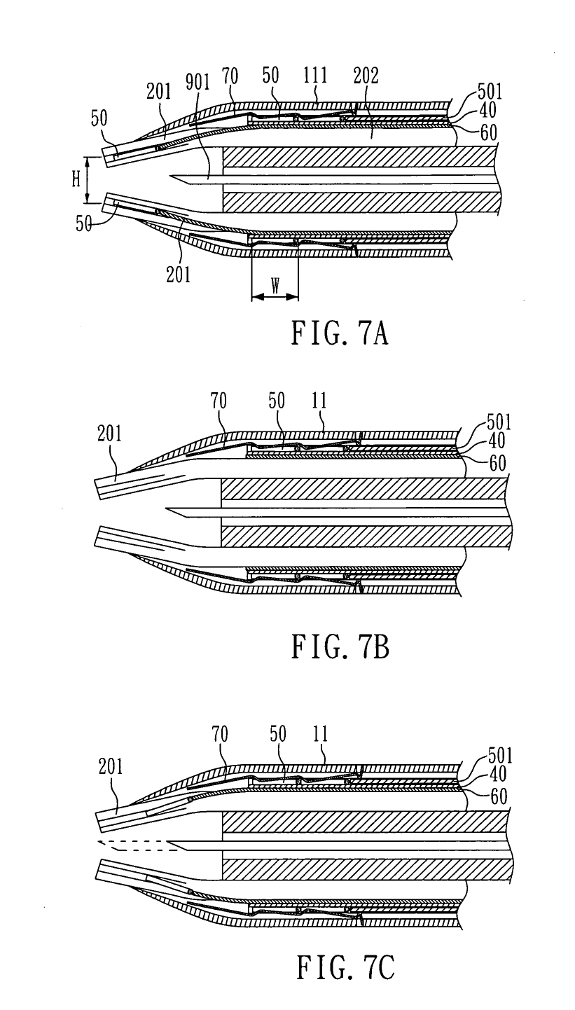

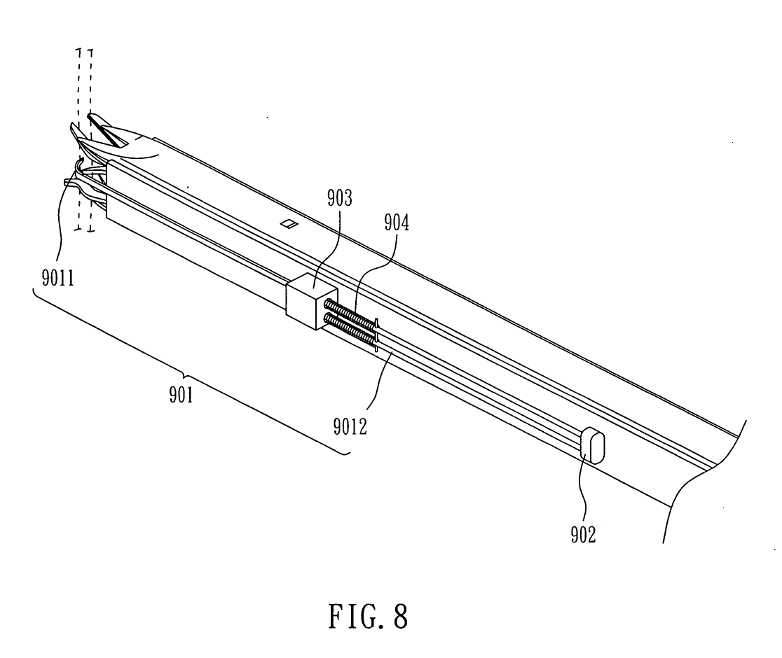

As shown in FIG. 2, the magazine unit 40 of the automatic clip applier of the present embodiment further comprises a temporary part 401, which has a slice 4011 for introducing the endmost ligating clip 50 in the magazine unit 40 to the clipping portion 201 of the jaw unit 20. Hereafter, the actuating mechanism for moving the ligating clip 50 to the clipping portion 201 of the jaw unit 20 is detailed described with reference to FIGS. 7A to 7C.

First, FIG. 7A is a perspective view showing the state that the automatic clip applier of the present embodiment is not operated. As shown in FIG. 7A, plural ligating clips 50 are disposed at the front ends of the tabs 501, and each clipping portion 201 is respectively disposed with a ligating clip 50.

Next, as shown in FIG. 7B and FIG. 4, when force is applied on the handling unit (not shown in the figure), the feeding unit 60 is drawn and moved in an opposite direction of the movement of the jaw housing 30. After the feeding unit 60 is moved, a sectional difference between the ligating clip 50 positioned at the temporary part 401 and the rest of the ligating clips 50 is generated.

Then, as shown in FIG. 7C and FIG. 4, when the force applied on the handling unit (not shown in the figure) is removed, the feeding unit 60 is moved back to its original state without any force applied thereon to push the ligating clip 50 positioned at the temporary part 401 of the magazine unit 40. Hence, the endmost ligating clip 50 can be moved to the clipping portion 201, and the automatic clip applier is in a state as shown in FIG. 7A.

In addition, as shown in FIG. 2 and FIG. 7A, the dispensing unit 70 is a saw-toothed (W-shaped) piece, and the pitch W of the dispensing unit 70 is the same as the length of the ligating clip 50. When the feeding unit 60 is drawn, the dispensing unit 70 with the saw-toothed shape can deliver a single ligating clip 50 to the temporary part 401 of the magazine unit 40. Hence, it is possible to deliver a single ligating clip 50 each time.

In addition, as shown in FIG. 2 and FIG. 7A, each jaw unit 20 of the automatic clip applier of the present embodiment further comprises an elongating part 202, which connects with the clipping portion 201 with an angle. Hence, the front end of each jaw units 20 is in a curved state. In the present embodiment, the jaw units 20 of the two clamping means bend inward, i.e. both the angles θ1, θ2 between the clipping portions 201 and the elongation parts 202 are smaller than 180°.

### Embodiment 2

The automatic clip applier of the present embodiment is similar to that of Embodiment 1, and the difference between the automatic clip applier of the present embodiment and that of Embodiment 1 is stated as follows.

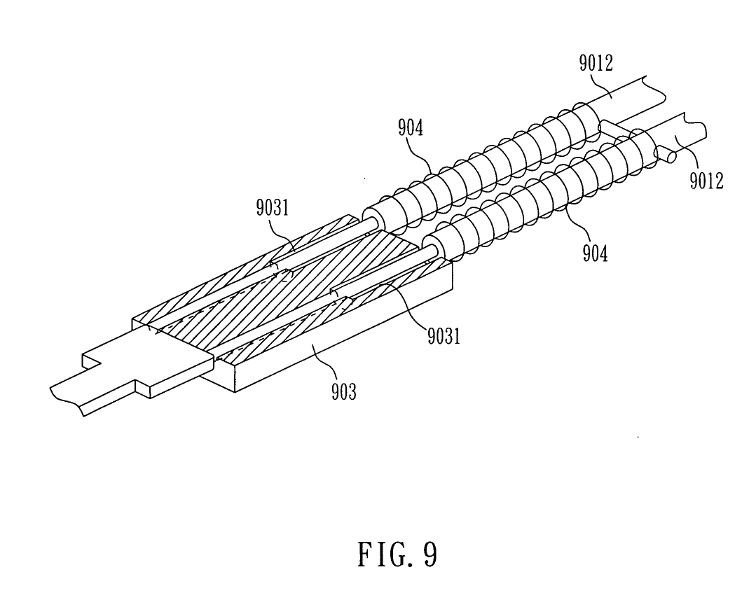

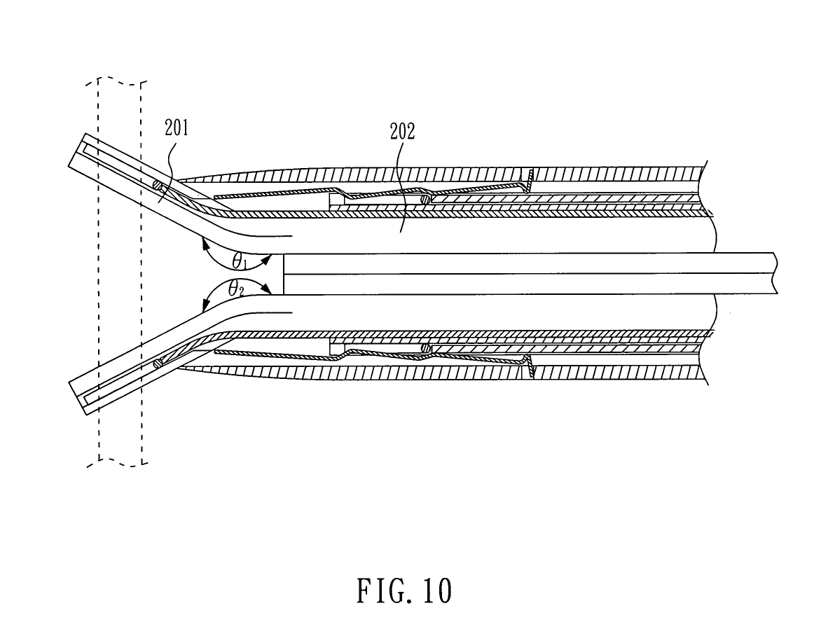

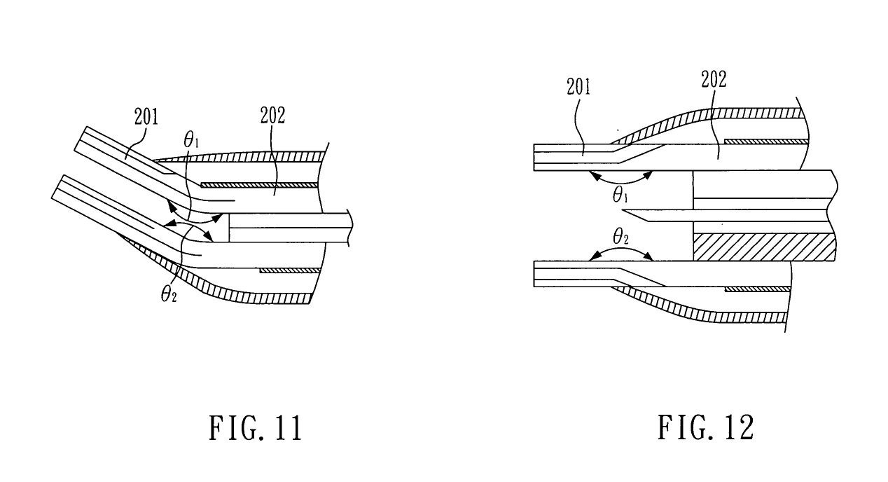

According to the automatic clip applier of the present embodiment, the cutting unit 901 and the advancing unit 902 are arranged on a side of one clamping means, as shown in FIG. 8. In addition, the automatic clip applier further comprises a fixing unit 903 arranged at the same side that the cutting unit 901 and the advancing unit 902 arranged thereon. The fixing unit 903 has a channel cavity 9031, and the cutting unit 901 is arranged in the channel cavity 9031 and moves with the channel cavity 9031, as shown in FIG. 9.

Furthermore, the cutting unit 901 of the automatic clip applier of the present embodiment further comprises a cutting component 9011 and a connecting component 9012, wherein the cutting component 9011 connects with the connecting component 9012, and the connecting component 9012 connects with the advancing unit 902. In addition, the automatic clip applier of the present embodiment further comprises an elastic element 904, wherein the connecting component 9012 is sheathed in the elastic element 904, and one end of the elastic element 904 connects with the fixing unit 903. In the present embodiment, the cutting component 9011 is a hook, as shown in FIG. 9.

In addition, as shown in FIG. 8, according to the automatic clip applier of the present embodiment, the jaw units 20 of the two clamping means bend outward, i.e. both the angles θ1, θ2 between the clipping portions 201 and the elongation parts 202 are larger than 180°.

### Embodiment 3

The automatic clip applier of the present embodiment is similar to that of Embodiment 2, except that the jaw unit 20 of one clamping means bends outward, and the jaw unit 20 of the other clamping mean bends inward. In other words, the angle θ1 between the clipping portion 201 and the elongation part 202 in one clamping means is larger than 180°, and the angle θ2 between the clipping portion 201 and the elongation part 202 in the other clamping means is smaller than 180°.

### Embodiment 4

The automatic clip applier of the present embodiment is similar to that of Embodiment 2, except that the jaw units 20 of both two clamping means are straight, i.e. both the angles θ1, θ2 between the clipping portions 201 and the elongation parts 202 are 180°.

Although the present invention has been explained in relation to its preferred embodiment, it is to be understood that many other possible modifications and variations can be made without departing from the scope of the invention as hereinafter claimed.

What is claimed is:

1. An automatic clip applier for clamping ligating clips, comprising:
a handling unit; and
two clamping means laminating with each other and connecting to the handling unit, and each clamping means respectively comprising:
a jaw unit containing a clipping portion to clamp a ligating clip;
a jaw housing containing a cavity, wherein the jaw unit is disposed in the cavity of the jaw housing;
a magazine unit for storing plural ligating clips;
a feeding unit for moving the endmost ligating clip from the magazine unit to the clipping portion of the jaw unit;
a dispensing unit for serially translating the endmost ligating clip to the end of the feeding unit; and
an operation unit connecting to the handling unit, the feeding unit, and the jaw housing, wherein the jaw housing is pushed and the feeding unit is drawn by operating the handling unit to move the jaw housing toward the clipping portion of the jaw unit to clamp the ligating clip positioning at the clipping portion, and to move the feeding unit in an opposite direction of the movement of the jaw housing, and the feeding unit moves back by restoring the handling unit to deliver the endmost ligating clip to the clipping portion of the jaw unit.

2. The automatic clip applier for clamping ligating clips as claimed in claim 1, further comprising a cutting unit and an advancing unit, wherein the cutting unit connects to the advancing unit, and the cutting unit is moved toward the clipping portions of the jaw units by operating the advancing unit.

3. The automatic clip applier for clamping ligating clips as claimed in claim 2, wherein the cutting unit comprises a cutting component and a connecting component, the cutting component connects with the connecting component, and the connecting component connects with the advancing unit.

4. The automatic clip applier for clamping ligating clips as claimed in claim 3, wherein the cutting unit and the advancing unit are disposed between the two clamping means.

5. The automatic clip applier for clamping ligating clips as claimed in claim 4, further comprising a channel cavity disposed between the two clamping means, wherein the advancing unit is placed inside the channel cavity.

6. The automatic clip applier for clamping ligating clips as claimed in claim 5, further comprising an elastic element, wherein the connecting component is sheathed into the elastic element, and one end of the elastic element connects to the advancing unit.

7. The automatic clip applier for clamping ligating clips as claimed in claim 2, wherein the cutting unit and the advancing unit are disposed on a side of one of the clamping means.

8. The automatic clip applier for clamping ligating clips as claimed in claim 7, further comprising a fixing unit disposed on the side of one of the clamping means, wherein the fixing unit has a channel cavity, and the advancing unit is placed inside the channel cavity.

9. The automatic clip applier for clamping ligating clips as claimed in claim 7, further comprising an elastic element, wherein the connecting component is sheathed into the elastic element, and one end of the elastic element connects to the fixing unit.

10. The automatic clip applier for clamping ligating clips as claimed in claim 3, wherein the cutting component is a blade.

11. The automatic clip applier for clamping ligating clips as claimed in claim 3, wherein the cutting component is a hook.

12. The automatic clip applier for clamping ligating clips as claimed in claim 1, wherein the operating unit comprises: a pull component and a push component, the pull component connects with the feeding unit and the handling unit, the push component connects with the jaw housing and the handling unit, the push component is pushed and the pull component is drawn by operating the handling unit, the push component is pushed to move the jaw housing toward the clipping portion of the jaw unit to clamp the ligating clip positioned at the clipping portion, the pull component is drawn to move the feeding unit in an opposite direction of the movement of the jaw housing, the feeding unit is moved back by restoring the handling unit to deliver the endmost ligating clip in the magazine unit to the clipping portion of the jaw unit.
